(19) United States
(12) Patent Application Publication
(10) Pub. No.: US 2005/0266040 A1
(43) Pub. Date: Dec. 1, 2005

(54) Medical devices composed of porous metallic materials for delivering biologically active materials

(76) Inventor: Brent Gerberding, San Jose, CA (US)

Correspondence Address: JONES DAY, 222 EAST 41ST ST, NEW YORK, NY, 10017, US

(21) Appl. No.: 10/857,740

(22) Filed: May 28, 2004

(51) Int. Cl. A61K 31/4745; A61K 38/18; A61K 31/337

(52) U.S. Cl. 424/423; 514/291; 514/449; 514/2

(57) ABSTRACT

A medical device, such as a stent, for delivering a biologically active material to body tissue of a patient, and a method for making such a medical device are described. The medical device has a coating layer on its surface. The coating layer includes a metal having a plurality of pores and a biologically active material dispersed in the pores. The pores are connected to the outer surface of the coating layer. The coating layer may be formed by applying a coating composition comprising two or more metals (such as a gold and silver) to the surface of the medical device and removing one of the metals to form the porous coating layer. This coating layer may be radiopaque, and may be substantially free of a polymeric material. The coating layer of the disclosed medical device has an increased surface area and, thus, can be loaded with a greater amount of biologically active material than a medical device without such coating.

### FIELD OF THE INVENTION

The invention relates generally to a medical device that is useful for delivering a biologically active material to the body tissue of a patient, and the method for making such a medical device. More particularly, the invention relates to medical devices having a coating layer comprising a metal having a plurality of surface-connected pores, and a biologically active material dispersed in the pores. The invention also relates to a method for providing such a coating layer on a medical device.

### BACKGROUND OF THE INVENTION

Medical devices, such as implantable stents, have been used to deliver biologically active material directly to body tissue of a patient, particularly for treating restenosis.

Recent studies have shown that higher doses of biologically active materials are more effective in treating restenosis. However, there are difficulties associated with using such medical devices to deliver a sufficient amount of biologically active materials to the body tissue to adequately treat the patient. These difficulties can be attributed to a number of factors. For example, there have been problems with adhering biologically active materials to the surface of the medical device.

In addition, there are also limitations with incorporating enough biologically material onto the medical device due to the limited surface area of the device. Specifically, the amount of biologically active material that can be applied to the stent is limited by the amount of surface area available to which the biologically active material can adhere. Thus, it is desirable to have a medical device or a coating for a medical device with a greater surface area so that a greater amount of biologically active material can be incorporated into or onto the medical device.

Another difficulty with applying biologically active materials, particularly in higher doses, to a medical device is preventing the biologically active material from releasing to the targeted tissue too rapidly, e.g., such as a burst effect. When higher doses of a biologically active material are applied to a medical device, it becomes more difficult to obtain a controlled release of the material. In addition, when biologically active materials are applied to a medical device, it is desirable to monitor the delivery and placement of the device in order to minimize any risk to the patient.

Accordingly, there is a need for a medical device that can deliver the desired dosage of a biologically active material. There is also a need for such a medical device that is radiopaque so that the medical device at the implantation site can be monitored. Furthermore, there is a need for a method of making a medical device with a greater surface area that can incorporate a sufficient amount of biologically active material that will release in a controlled manner over time from the medical device.

### SUMMARY OF THE INVENTION

These and other objectives are accomplished by the present invention. The present invention, in one embodiment, provides a coated medical device for delivering a biologically active material to body tissue of a patient. The coated medical device comprises a medical device having a surface; and a coating layer disposed on at least a portion of the surface. The coating layer comprises an outer surface and also comprises a biocompatible metal having a plurality of pores that are connected to the surface of the coating layer, i.e., surface-connected. A biologically active material is contained in the pores. The pores are preferably micropores or nanopores.

In another embodiment, a method of making a coated medical device for delivering a biologically active material to the body tissue of a patient is disclosed. Specifically, the method of the present invention comprises providing a medical device having a surface and applying to at least a portion of the surface a coating composition comprising a first metal, which is biocompatible. A coating layer of the coating composition is formed on the surface of the medical device comprising an outer surface. The coating layer comprises the first metal having a plurality of pores that are connected to the outer surface of the coating layer. A biologically active material is dispersed or simply placed in the pores. The coating composition may further comprise a second metal and the coating layer may be formed by removing the second metal, such as by applying an acid to the coating composition. The first metal may comprise gold and the second metal may comprise silver. A coated medical device made according to the method of the present invention is also disclosed.

In yet another embodiment, a method of making a radiopaque coated medical device for delivering a biologically active material to the body tissue of a patient is disclosed. The method of the present invention comprises providing a medical device having a surface; and applying to the surface a coating composition comprising a first metal and a second metal. The second metal is removed to form a coating layer on the surface, wherein the coating layer comprises an outer surface. Also, the coating layer comprises the first metal having a plurality of pores that are connected to the outer surface of the coating layer. A biologically active material is dispersed in the pores.

The present invention has the advantage of having an increased surface area due to the plurality of pores in the coating layer of the device that are connected to the outer surface of the coating layer. By being connected to the outer surface of the coating layer, the pores facilitate the release of the biologically active material from the pores. Thus, the present invention provides for a medical device that allows greater amounts of biologically active material to be loaded on the medical device due to the increased surface area. In addition, the present invention provides for a coated medical device that allows the biologically active material to be dispersed deeper in the device or coating layer of the device and be released more slowly or in a controlled manner over time. The present invention also provides for such a medical device with radiopacity.

### BRIEF DESCRIPTION OF THE DRAWINGS

The present invention will be explained with reference to the following drawings.

FIG. 1 is a cross-sectional view of a medical device having a coating of the present invention.

FIG. 2a is a scanning electron micrograph of a cross-section of a film of gold having surface-connected pores.

FIG. 2b is a scanning electron micrograph of a plan view of a film of gold having surface-connected pores.

### DETAILED DESCRIPTION

The coated medical device of the present invention comprises a medical device having a surface. Suitable medical devices include, but are not limited to, stents, surgical staples, catheters, such as central venous catheters and arterial catheters, guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, extra-corporeal devices such as blood oxygenators, blood filters, hemodialysis units, hemoperfusion units or plasmapheresis units.

Medical devices which are particularly suitable for the present invention include any stent for medical purposes, which are known to the skilled artisan. Suitable stents include, for example, vascular stents such as self-expanding stents and balloon expandable stents. Examples of self-expanding stents are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al.

The framework of the suitable stents may be formed through various methods as known in the art. The framework may be welded, molded, laser cut, electro-formed, or consist of filaments or fibers which are wound or braided together in order to form a continuous structure.

The medical devices suitable for the present invention may be fabricated from ceramic, polymeric and/or metallic materials. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

In the present invention, at least a portion of the surface of the medical device is coated with a coating layer. The coating layer may be substantially free of polymeric materials. This coating layer has an outer surface, which is the surface opposite the surface of the coating layer that is nearest to the medical device surface. The coating layer comprises a first metal having a plurality of pores, wherein the metal is biocompatible. Suitable first metals include, but are not limited to, gold, platinum, stainless steel, tantalum, titanium, iridium, molybdenum, niobium, palladium, or chromium. A preferred first metal is gold.

Preferably, the first metal is a radiopaque material. Including a radiopaque material may be desired so that the medical device is visible under X-ray or fluoroscopy. Suitable first metals that are radiopaque include gold, tantalum, platinum, bismuth, iridium, zirconium, iodine, titanium, barium, silver, tin, alloys of these metals, or similar materials.

Moreover, the pores in the first metal are connected to or in communication with the outer surface of the coating layer. Having the pores connected to the surface facilitate the biologically active materials placed in the pores to be released, e.g., eluted, from the pores. Also the pores may be discrete, interconnected, or disposed in a pattern. In addition, the pores may have any shape or size, but are preferably micropores or nanopores. Additionally, the pores can be shaped like channels, void pathways or microscopic conduits.

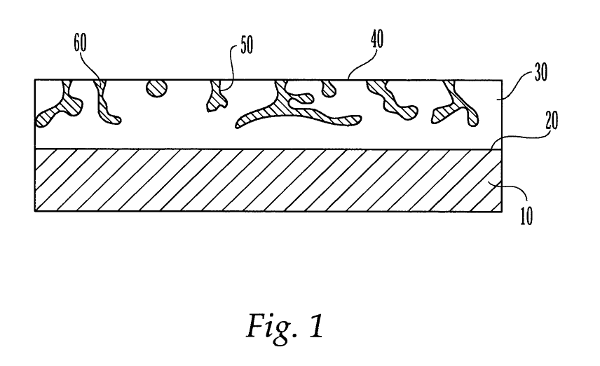

FIG. 1 shows a cross-sectional view of a section of a medical device comprising the coating of the present invention. The medical device 10 comprises a surface 20. A coating layer 30 is disposed on at least a portion of the surface of the medical device 20. The coating layer comprises an outer layer 40. The coating layer has a plurality of pores 50 that are connected to the outer surface of the coating layer 40. Biologically active materials 60 are in the surface-connected pores 50. As shown in this figure, the pores can have different shapes and sizes.

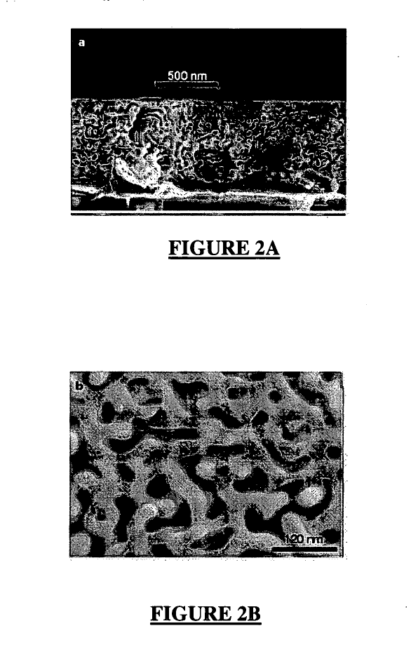

FIGS. 2a and 2b are scanning electron micrographs (SEM) of a gold film having pores that are connected to the outer surface of the film. FIG. 2a is a cross-sectional view of the gold film having a plurality of nanopores. FIG. 2b is a plan view of the gold film which shows that the pores are connected to the outer surface of the film.

A biologically active material is contained or dispersed in the pores in the coating layer. The term “biologically active material” encompasses therapeutic agents, such as drugs, and also genetic materials and biological materials. Suitable genetic materials include DNA or RNA, such as, without limitation, DNA/RNA encoding a useful protein and DNA/RNA intended to be inserted into a human body including viral vectors and non-viral vectors as well as anti-sense nucleic acid molecules such as DNA, RNA and RNAi. Suitable viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, sketetal myocytes, macrophage), replication competent viruses (e.g., ONYX-015), and hybrid vectors. Suitable non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP 1017 (SUPRATEK), lipids or lipoplexes, nanoparticles and microparticles with and without targeting sequences such as the protein transduction domain (PTD).

Suitable biological materials include cells, yeasts, bacteria, proteins, peptides, cytokines and hormones. Examples of suitable peptides and proteins include growth factors (e.g., FGF, FGF-1, FGF-2, VEGF, Endothelial Mitogenic Growth Factors, and epidermal growth factors, transforming growth factor α and β, platelet derived endothelial growth factor, platelet derived growth factor, tumor necrosis factor α, hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Biologically active material also includes non-genetic therapeutic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, tacrolimus, everolimus, amlodipine and doxazosin; anti-inflammatory agents such as glucocorticoids, betamethasone, dexamethasone, prednisolone, corticosterone, budesonide, estrogen, sulfasalazine, rosiglitazone, mycophenolic acid and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides; DNA demethylating drugs such as 5-azacytidine, which is also categorized as a RNA or DNA metabolite that inhibit cell growth and induce apoptosis in certain cancer cells; vascular cell growth promotors such as growth factors, Vascular Endothelial Growth Factors (FEGF, all types including VEGF-2), growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents, vasodilating agents, and agents which interfere with endogenous vasoactive mechanisms; anti-oxidants, such as probucol; antibiotic agents, such as penicillin, cefoxitin, oxacillin, tobranycin, a macrolide such as everolimus and rapamycin (sirolimus); angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril, statins and related compounds.

Preferred biologically active materials include anti-proliferative drugs such as steroids, vitamins, and restenosis-inhibiting agents. Preferred restenosis-inhibiting agents include microtubule stabilizing agents such as Taxol, paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other preferred biologically active materials include nitroglycerin, nitrous oxides, antibiotics, aspirins, digitalis, and glycosides as well as immunosuppressants such as rapamycin (sirolimus).

The amount of biologically active material can be adjusted to meet the needs of the patient. In general, the amount of the biologically active material used may vary depending on the application or biologically active material selected. One of skill in the art would understand how to adjust the amount of a particular biologically active material to achieve the desired dosage or amount.

The coating layer may be any thickness, but preferably has a thickness of about 1.0 to about 50 microns. A thicker coating layer may be preferred to incorporate greater amounts of biologically active material. In addition, a thicker coating layer will allow the biologically active material to penetrate deeper into the coating layer and release from the pores in the coating layer more slowly over time.

To make a medical device coating of the present invention, a coating composition is first applied to at least a portion of the surface of a medical device. The coating composition comprises a first metal, which is biocompatible, as described above. Also, the coating composition includes a second metal. Like the first metal, the second metal is preferably biocompatible. Suitable second metals include, but are not limited to, silver, gold, aluminum, tantalum, platinum, bismuth, iridium, selenium, sulfur, tin, zirconium, iodine, titanium, barium, chromium, calcium, copper, magnesium, potassium, iron, sodium, and zinc. The second metal can be in the form of particles such as hollow spheres or chopped tubes of various sizes. When the second metal is removed, as discussed below, the size of the pores formed will be determined by the size of the second metal particles. For example, if a hollow sphere of the second metal is removed, the size of the cavity of the sphere will determine the size of the pore formed. Preferably, the first and second metals are different metals. The two metals can form an alloy such as a gold/silver alloy, where gold is the first metal and silver is the second metal. Also, the two metals can be in the form of a mechanical mixture or a composite. As discussed below, the second metal is removed to form the pores. Thus, the metals should have different chemical or physical properties to facilitate removal of the second metal. For example, the second metal should be more electrochemically active, e.g., less corrosion-resistant than the first metal.

In another embodiment, the second metal should have a lower melting point than the first metal. In yet another embodiment, the second metal should have a higher vapor pressure than the first metal. Also, in another embodiment, the second metal is more susceptible to being dissolved in a chosen solvent than the first metal.

The coating composition is applied to at least a portion of the surface of the medical device by any suitable method such as, but not limited to, dipping, spraying, painting, electroplating, evaporation, plasma-vapor deposition, cathodic-arc deposition, sputtering, ion implantation, electrostatically, electroplating, electrochemically, a combination of the above, or the like.

After the coating composition is applied to the surface of the medical device, a coating layer having a plurality of pores is formed from the coating composition. The coating layer is formed by any suitable method. For example, when the coating composition comprises a first metal and a second metal, the coating layer is formed by removing the second metal by any suitable method as known by one of ordinary skill in the art.

For example, the second metal may be removed from the first metal by a dealloying process such as selective dissolution of the second metal. In this method, the coating composition is exposed to an acid which removes the second metal. Thus, the first metal is preferably one that will not dissolve when exposed to the acid, while the second metal is one that will dissolve. Any suitable acid can be used to remove the second metal. One of ordinary skill in the art would recognize the appropriate concentration and reaction conditions to use to remove the second metal. For example, if the second metal is silver, nitric acid may be used at a concentration of up to 35% and a temperature up to 120° F. Also, a nitric acid and sulfuric acid mixture (95%/5%) immersion process at 80° F. may be used. The reaction conditions may be varied to vary the geometry, distribution, and depth of the coating layer.

In an embodiment, the first metal and second metal formed a two-phase structure. One phase comprises mostly of the second metal. This phase is preferentially chemically milled away. The morphology of this phase may be optimized for chemical removal by heat treatment. For example, a long needle-like or interdendritic phase morphology might produce a better network for therapeutic agent infiltration and release as compared to a globular phase morphology.

Alternatively, the second metal can be removed anodically. For example, silver may be removed from the coating composition anodically using a dilute nitric acid bath comprising up to 15% nitric acid, wherein the anode is the plated stent, and the cathode is platinum. Voltages up to 10V DC can be applied across the electrodes. The bath chemistry, temperature, applied voltage, and process time may be varied to vary the geometry, distribution, and depth of the coating layer. In another example, a Technic Envirostrip Ag 10-20 amps per square foot may be used with a stainless steel cathode.

Furthermore, if the second metal has a lower melting point than the first metal, the device coated with the first and second metal can be heated to a temperature such that the second metal becomes a liquid and is removable from the solid first metal. Examples of suitable metals for such a process include one of the higher melting point first metals: platinum, gold, stainless steel, titanium, tantalum, and iridium, in combination with one of the lower melting point second metals such as: aluminum, barium, and bismuth.

In another embodiment, the second metal has a higher vapor pressure than the first metal such that when the device coated with the first and second metal is heated under vacuum the second metal becomes vaporized and is removed from the solid first metal. Exemplary metals for this technique include one of the first metals: platinum, gold, titanium, tantalum, iridium, molybdenum, niobium, and palladium, in combination with one of the second metals: chromium, aluminum, barium, bismuth, calcium, copper, magnesium, and potassium.

In an embodiment, a fine metal powder or beads may be attached to the surface of the coating. A binder may be used to glue the metal particles onto the surface. The metal particles may also be heated to a temperature and diffusion-bonded together. A braze alloy and diffusion bonding activator may be included in the binder to promote diffusion bonding at a lower temperature range than would be deleterious to the coating. For further description of dealloying processes and the formation of a nanoporous structure, see Erlebacher, et. al., “Evolution of Nanoporsity in Dealloying”, Nature, vol. 410, Mar. 22, 2001, pp. 450-453.

After the second metal is removed from the coating composition, a coating layer comprising an outer surface and the first metal having a plurality of pores remains on the surface of the medical device. The pores are connected to the outer surface of the coating. A biologically active material is dispersed in the pores of the coating layer by any suitable method, such as, but not limited to dip coating, spray coating, spin coating, plasma deposition, condensation, electrochemically, electrostatically, evaporation, plasma vapor deposition, cathodic arc deposition, sputtering, ion implantation, or use of a fluidized bed. In order to disperse the molecules of the biologically active material in the pores, it may be necessary to modify the size of the pores in the coating layer. The pore size may be modified by any suitable method, such as heat treatment.

In an alternative method, the coating layer having a plurality of pores may be formed on the coating using vacuum plasma spraying on the coating comprising a first metal with process parameters that promote the formation of porosity. These process parameters are known to those skilled in the art. The pore size could be varied by how much entrapped gas is present in the coating.

A medical device, such as a stent, according to the present invention can be made to provide desired release profile of the biologically active material. For example, the amount of coating composition applied to the surface of the medical device and the technique and reaction conditions used in forming the coating layer can be adjusted to vary the thickness and porosity of the coating layer. By increasing the thickness and/or porosity a greater amount of biologically active material may be dispersed in the coating layer.

The medical devices and stents of the present invention may be used for any appropriate medical procedure. Delivery of the medical device can be accomplished using methods well known to those skilled in the art.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

1. A coated medical device for delivering a biologically active material to body tissue of a patient comprising:
a medical device having a surface;
a coating layer disposed on at least a portion of the surface, wherein the coating layer comprises an outer surface and a biocompatible metal having a plurality of pores that are connected to the outer surface of the coating layer;
and a biologically active material contained in the pores.

2. The medical device of claim 1, wherein the pores are nanopores.

3. The medical device of claim 1, wherein the coating layer is substantially free of a polymeric material.

4. The medical device of claim 1, wherein the medical device is a stent.

5. The medical device of claim 1, wherein the metal comprises gold, platinum, stainless steel, titanium, tantalum, iridium, molybdenum, niobium, palladium or chromium.

6. The medical device of claim 1, wherein the biologically active material comprises an anti-thrombogenic agent, anti-angiogenesis agent, anti-proliferative agent, antibiotic agent, growth factor, immunosuppressant, radiochemical, or combination thereof.

7. The medical device of claim 6, wherein the anti-proliferative agent comprises paclitaxel, paclitaxel analogues or paclitaxel derivatives.

8. The medical device of claim 6, wherein the antibiotic agent comprises a macrolide such as sirolimus and everolimus.

9. The medical device of claim 1, wherein the metal is radiopaque.

10. A method of making a coated medical device for delivering a biologically active material to the body tissue of a patient, the method comprising:
providing a medical device having a surface;
applying to at least a portion of the surface a coating composition comprising a first metal which is biocompatible to form a coating layer on at least a portion of the surface, wherein the coating layer comprises an outer surface and the first metal has a plurality of pores connected to the outer surface of the coating layer; and
placing a biologically active material in the pores.

11. The method of claim 10, wherein the pores are nanopores.

12. The method of claim 10, wherein the coating layer is substantially free of a polymeric material.

13. The method of claim 10, wherein the medical device is a stent.

14. The method of claim 10, wherein the first metal comprises gold, platinum, stainless steel, titanium, tantalum, iridium, molybdenum, niobium, palladium or chromium.

15. The method of claim 10, wherein the biologically active material comprises an anti-thrombogenic agent, anti-angiogenesis agent, anti-proliferative agent, antibiotic agent, growth factor, immunosuppressant, radiochemical or combination thereof.

16. The method of claim 15, wherein the anti-proliferative agent comprises paclitaxel, paclitaxel analogues or paclitaxel derivatives.

17. The method of claim 15, wherein the antibiotic agent comprises a macrolide such as sirolimus and everolimus.

18. The method of claim 10, wherein the first metal is radiopaque.

19. The method of claim 10, wherein the coating composition further comprises a second metal, and the coating layer is formed by removing the second metal.

20. The method of claim 19, wherein the second metal is removed by exposing the second metal to an acid.

21. The method of claim 19, wherein the first metal comprises gold and the second metal comprises silver.

22. A coated medical device made according to the method of claim 10.

23. A method of making a radiopaque coated medical device for delivering a biologically active material to the body tissue of a patient, the method comprising:
providing a medical device having a surface;
applying to the surface a coating composition comprising a first metal which is biocompatible and a second metal;
removing the second metal to form a coating layer on the surface, wherein the coating layer comprises an outer surface and the coating layer comprises the first metal having a plurality of pores that are connected to the outer surface of the coating layer; and
placing a biologically active material in the pores.

24. The method of claim 23, wherein the pores are nanopores.

25. The method of claim 23, wherein the first metal comprises gold and the second metal comprises silver.

26. The method of claim 23, wherein the first metal comprises gold, platinum, stainless steel, titanium, tantalum, iridium, molybdenum, niobium, palladium or chromium.

27. The method of claim 23, wherein the second metal comprises silver, aluminum, barium, bismuth, chromium, calcium, copper, magnesium, potassium, iron, sodium, iridium, selenium, sulfur, tin or zinc.
